(19) United States
(12) Patent Application Publication
(10) Pub. No.: US 2012/0283771 A1
(43) Pub. Date: Nov. 8, 2012

(54) Craniotomy Plugs

(75) Inventors: James D. Ralph, Bethlehem, PA (US); Thomas N. Troxell, Pottstown, PA (US); Mark Michels, Glen Mills, PA (US)

(73) Assignee: BIODYNAMICS, LLC, Hackensack, NJ (US)

(21) Appl. No.: 13/463,669

(22) Filed: May 3, 2012

Related U.S. Application Data
(60) Provisional application No. 61/482,039, filed on May 3, 2011.

(51) Int. Cl. A61B 17/03

(52) U.S. Cl. 606/213

(57) ABSTRACT

Described herein is a cranial plug for a burr hole or a cranial perforation made in a skull during brain surgery, the cranial plug being flexible and having a bottom portion and side walls extending upwards from the bottom portion, the bottom portion and the side walls formed in a concave or substantially concave shape, or alternatively, forming a concave or substantially concave shape when inserted into a burr hole or cranial perforation.

### CLAIM OF PRIORITY

This application claims priority to U.S. Provisional Application No. 61/482,039 filed May 3, 2011, the disclosure of which is hereby incorporated by reference in its entirety.

### FIELD OF THE INVENTION

The invention relates to cranial plugs used to fill small openings in the cranium or the burr holes made to facilitate cutting out a skull flap. In a further aspect, the invention relates to plugs and methods which enhance bone growth and the consequent healing of the skull flap and the skull.

### BACKGROUND OF THE INVENTION

Surgical access to the brain for neurosurgical procedures is created by removing a portion of the patient's skull, a procedure termed a craniotomy. The craniotomy is determined by the location of the pathology within the brain, the safest/easiest access route and the degree of exposure required for the procedure. Once the location is determined, the first step is to create an initial perforation of the full thickness of the skull. Special skull perforators are available to create perfectly round holes but most surgeons simply use a rounded, end-cutting burr to create the perforation. Typically the perforation is in the range of about 11-15 millimeters (mm) in diameter. A surgeon may choose to create more than one perforation around the perimeter of the planned craniotomy. Some surgeons prefer a single perforation and others use more than one, but there is no standard number. Once this hole is created, it allows the insertion of a rotary powered surgical instrument (e.g., a craniotome) which is used to create a continuous cut (kerf) around the perimeter of the craniotomy. This kerf begins and ends at the perforation when there is one perforation or it runs from one perforation to another when more than one perforation is made in the skull. The kerf is made with a side cutting burr which is shielded from the dura (outer covering of the brain) by a foot plate on the craniotome. The foot plate extends below and forward of the cutting burr and the surgeon keeps the tip of the foot plate in contact with the inner surface of the skull as he performs the craniotomy. The typical kerf is made freehand with an approximately 2 mm diameter burr. The shape of the craniotomy is therefore highly variable and the kerf is not always oriented perpendicular to the skull. The kerf may be larger than 2 mm in some areas as well. Over the course of the kerf, the skull thickness will vary, typically over the range of 3-8 mm in adults.

Once the cut is complete, the skull flap is removed from the skull and placed on the sterile back table for reinsertion at the end of the procedure. After completion of the soft tissue surgery (typically 1-6 hours), the skull flap is inserted back into the craniotomy and fixated to prevent movement and restore the original contour of the skull. The surgeon may bias the skull flap toward one side or another to create bone-to-bone contact in a particular area or he may leave a gap around the entire flap. The scalp is then closed and the patient is sent to the neurosurgical intensive care unit for recovery.

If complications develop while the patient is in the hospital, there may be the need for emergency access to the brain through the craniotomy site. In addition, some patients may return for subsequent craniotomies in the same region, particularly in cases of recurrent tumors. Postoperative imaging studies (MRI or CT) are generally conducted on all patients. There is no clear evidence that the skull flap ever completely heals (solid bony union) in adults. It is more likely that a combination of new bone formation and fibrous connective tissue fills the gap between the skull and the skull flap.

From a surgeon's perspective, the method of reattaching the bone flap must be safe, simple to use, be rapidly applied, permit emergent re-entry, not interfere with postoperative imaging studies, provide stable fixation and have an acceptably low profile. The ideal method would result in complete fusion of the bone flap to the native skull with no long term evidence of prior surgery.

Current methods of reattaching the skull flap include drilling a series of small holes in the edge of the skull and the edge of the flap. Sutures are then passed through the corresponding holes and the flap is secured back into the skull opening from which it was taken. Because the fit is not exact due to the material removed by the craniotome, the flap can sag and sit slightly below the surface of the skull resulting in a depressed area that is obvious through the skin.

Another common reattachment method substitutes stainless steel wire for the suture material and fewer holes are used. There is still the risk of a cosmetically objectionable depressed area resulting. Metallic cranial fixation is (generally) only ever removed if it becomes symptomatic or if it interferes with subsequent surgeries.

More recently, surgeons have begun to use the titanium micro plates and screws that were developed for internal fixation of facial and finger bones. While this method results in a more stable and cosmetic result, it is relatively expensive, does not insure fusion and leaves foreign bodies at the surgical site.

All of these methods take ten minutes to one hour of additional surgery after the soft tissue (brain) surgery.

There is another method in which a titanium rivet (or clamp) is placed inside the skull with the stem of the rivet (clamp) passing between the skull and the flap. A large “pop rivet” type tool is used to force an upper titanium button down over the stem of the rivet, locking the flap and the skull in place between the upper and lower buttons. Three or four of these rivets and buttons are used to secure the flap in place. This method can be faster than other methods and less expensive than the titanium plates, but more expensive than sutures or wires. Just as with titanium plates and screws, fusion is not assured and foreign bodies remain in the patient.

According to the present invention we have developed cranial plugs for burr holes or cranial perforations. The cranial plugs of the invention are secure and cosmetically acceptable. The plugs also can enhance bone growth in a manner which causes healing by means of bone-to-bone reattachment of the skull flap to the skull.

### SUMMARY OF THE INVENTION

The cranial plugs of the invention are used to plug small circular openings in the skull such as those made by a surgeon to gain access for surgery or to insert a cutting instrument such as a craniotome to cut out a skull flap. Sometimes more than one small hole is made in the skull to facilitate cutting out a skull flap and, following surgery, the cranial plugs can be used to fill each of those holes, sometimes referred to as burr holes. The cranial plugs can be used by themselves, or in combination with fasteners, such as those described in U.S. Pat. No. 8,080,042, the disclosure of which is hereby incorporated by reference in its entirety. The cranial plug design described herein also can be filled or partially filled with medication, bone paste, bone growth enhancers and the like.

The cranial plugs of the present invention do not contain a top flange—they are simply inserted into the burr hole, the sidewalls of the plug conform to the sidewalls of the burr hole and act as spring fingers to hold the plug in place and, in certain embodiments, provide openings through which the bone paste can move sideways to contact the bone. This also provides pathways for fusion.

The present invention is therefore directed to a cranial plug for a burr hole or a cranial perforation made in a skull during brain surgery, the cranial plug having a bottom portion and side walls extending upwards from the bottom portion.

In certain embodiments, the present invention is directed to a cranial plug for a burr hole or a cranial perforation made in a skull during brain surgery, the cranial plug having a bottom portion and side walls extending upwards from the bottom portion, the bottom portion and the side walls forming a concave or substantially concave shape.

In other embodiments, the present invention is directed to a cranial plug for a burr hole or a cranial perforation made in a skull during brain surgery, the cranial plug having a bottom portion and side walls, wherein the cranial plug is substantially flat prior to insertion into a burr hole or a cranial perforation and wherein the cranial plug is concave or substantially concave, with the side walls extending upwards from the bottom portion when the cranial plug is inserted into a burr hole or a cranial perforation.

As used herein, the term “substantially concave” shall mean a rounded structure or a structure having a flat or substantially flat bottom with sides extending upward.

### BRIEF DESCRIPTION OF THE DRAWINGS

The appended drawings are not intended to illustrate every embodiment of the invention but they are representative of embodiments within the principles of the invention. The drawings are for illustrative purposes and are not drawn to scale.

FIG. 1 is a perspective view of an embodiment of a cranial plug of the present invention.

FIG. 2 is a perspective view of an embodiment of a cranial plug of the present invention.

FIG. 3 is a side view of an embodiment of a cranial plug of the present invention.

FIG. 4 is bottom view of an embodiment of a cranial plug of the present invention.

FIG. 5 is an alternate side view of an embodiment of a cranial plug of the present invention.

FIG. 6 is a perspective top view of an embodiment of a cranial plug of the present invention.

FIG. 7 is a top view of an embodiment of a cranial plug of the present invention.

FIG. 8 is a perspective view of an embodiment of a cranial plug of the present invention.

FIG. 9 is a view of an embodiment of a cranial plug of the present invention when inserted into a burr hole.

FIG. 10 is an embodiment of a cranial plug of the present invention when the plug is substantially flat prior to insertion into a burr hole.

FIG. 11 is a perspective view of an alternate embodiment of a cranial plug of the present invention.

FIG. 12 is a perspective view of an alternate embodiment of a cranial plug of the present invention.

FIG. 13 is a perspective view of an alternate embodiment of a cranial plug of the present invention.

FIG. 14 is an embodiment of a cranial plug of the present invention when the plug is substantially flat prior to insertion into a burr hole.

### DETAILED DESCRIPTION

The cranial plugs of the present invention may be made, in part or in its entirety, with bioabsorbable material. The term “bioabsorbable material” as used herein includes materials which are partially or completely bioabsorbable in the body.

Suitable bioabsorbable materials include collagen, polyglycolide, poly(lactic acid), copolymers of lactic acid and glycolic acid, poly-L-lactide, poly-L-lactate; crystalline plastics such as those disclosed in U.S. Pat. No. 6,632,503 which is incorporated herein by reference; bioabsorbable polymers, copolymers or polymer alloys that are self-reinforced and contain ceramic particles or reinforcement fibers such as those described in U.S. Pat. No. 6,406,498 which is incorporated herein by reference; bioresorbable polymers and blends thereof such as described in U.S. Pat. No. 6,583,232 which is incorporated herein by reference; copolymers of polyethylene glycol and polybutylene terephthalate, and the like. The foregoing list is not intended to be exhaustive. Other bioabsorbable materials can be used based upon the principles of the invention as set forth herein. Some of the most common include Poly-L-lactic acid (PLLA) Poly-DL-lactic acid (PDLLA) Polyglycolic acid (PGA) Polydioxanone (PDS) Polyorthoester (POE) Poly-C-capralactone (PCL).

Bioactive materials can be admixed with the bioabsorbable materials, impregnated in the bioabsorbable materials and/or coated on the outer surface thereof. Bioactive materials, including natural and/or synthetic materials, also can be used to fill cavities in the cranial plugs. These materials can include, for example, bioactive ceramic particles, bone chips or paste, platelet rich plasma (PRP), polymer chips, synthetic bone cement, autologous materials, allograft, cadaveric materials, xenograft, nanoparticles, nanoemulsions and other materials employing nanotechnology, capsules or reinforcement fibers. And they can contain, for example, antimicrobial fatty acids and related coating materials such as those described in Published U.S. Patent Publication No. 2004-0153125; antibiotics and antibacterial compositions; immunostimulating agents; tissue or bone growth enhancers and other active ingredients and pharmaceutical materials known in the art.

The cranial plugs of the invention which are made with bioabsorbable material can be made by molding, extrusion, heat shrinking or coating the bioabsorbable material on a base which has been provided with attachment means such as those described in Published U.S. Patent Publication No. 2006-0142772 which is incorporated herein by reference. When the bioabsorbable material will have functional mechanical properties which are not made from the base material, the bioabsorbable material can be molded onto the base in the desired shape. Alternatively, the bioabsorbable material also can be coated, shrink wrapped or molded onto the base. If necessary, the bioabsorbable material can be machined to the desired shape and/or dimensions.

As will be apparent to those skilled in the art, the sizes of the plugs of the invention can be varied to meet their intended applications. The shapes can take various forms in addition to those illustrated without deviating from the principles of the invention. And the sizes, lengths and widths can be varied for particular applications within the principles of the invention set forth herein.

When the plug is inserted into the burr hole, at least a portion of the plug would cover the outer layer of the brain, the dura. The intent is to create a “floor” in the opening to aid in containment of autologous and other bioactive substances with the goal of creating fusion across the burr hole. Without this “floor”, these substances could easily be pushed beneath the skull, into the space between the dura and inside of the skull. This would result in bioactive substances where they are not intended and it would also force the surgeon to use a larger volume of these materials than is necessary. The latter is a concern because bioactive substances are costly, or when harvested autologously, are available in limited quantity.

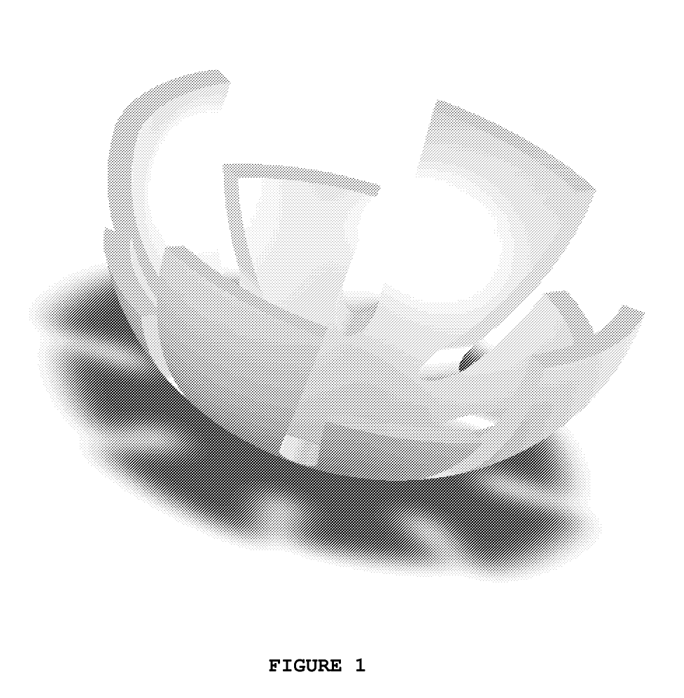

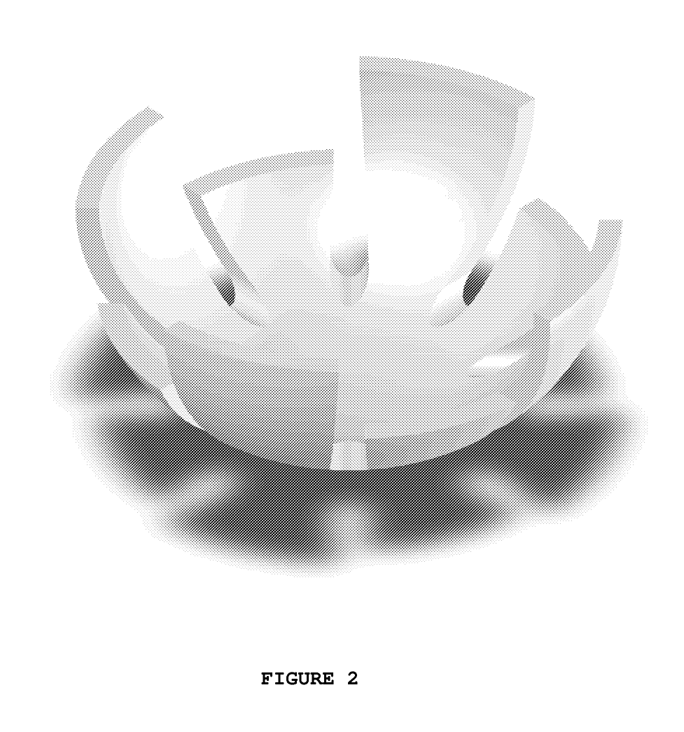

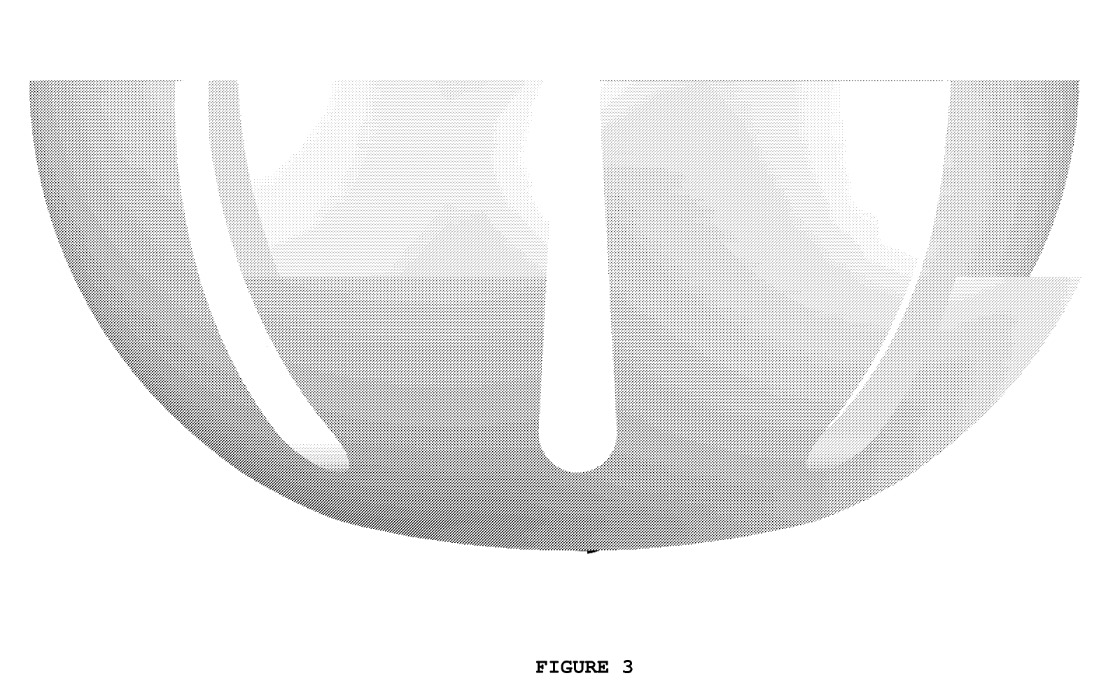

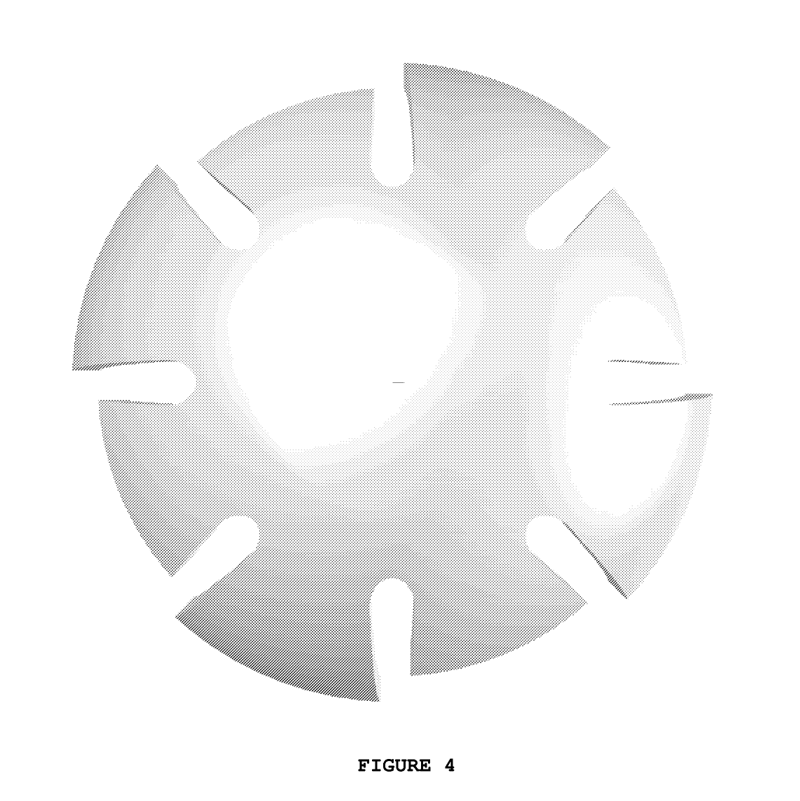

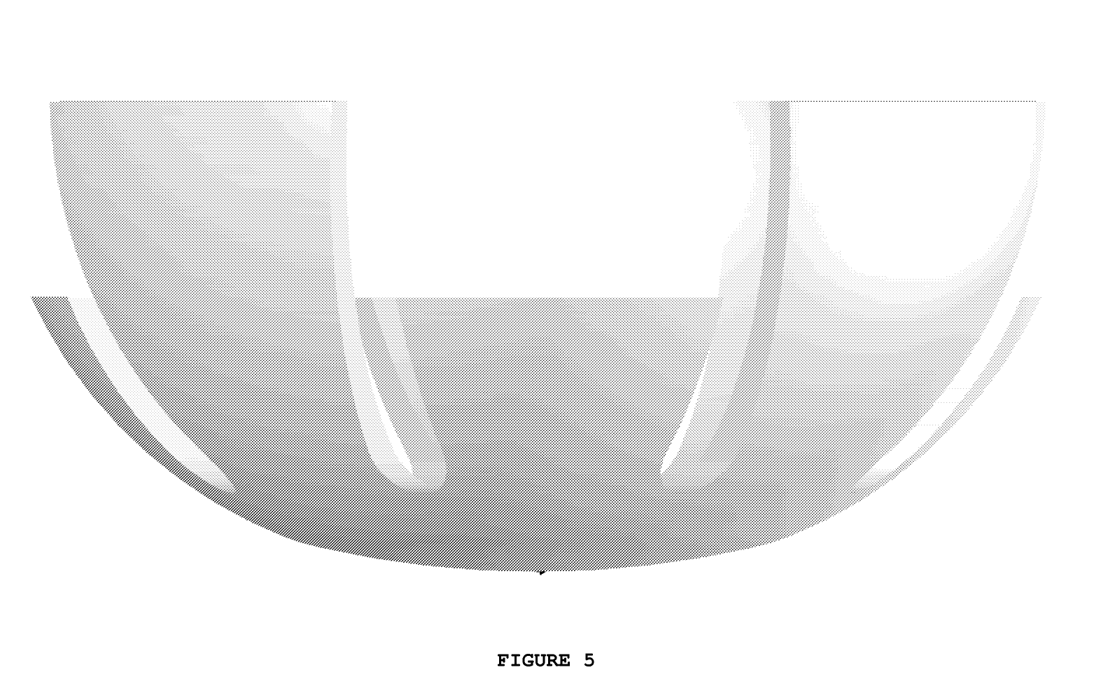

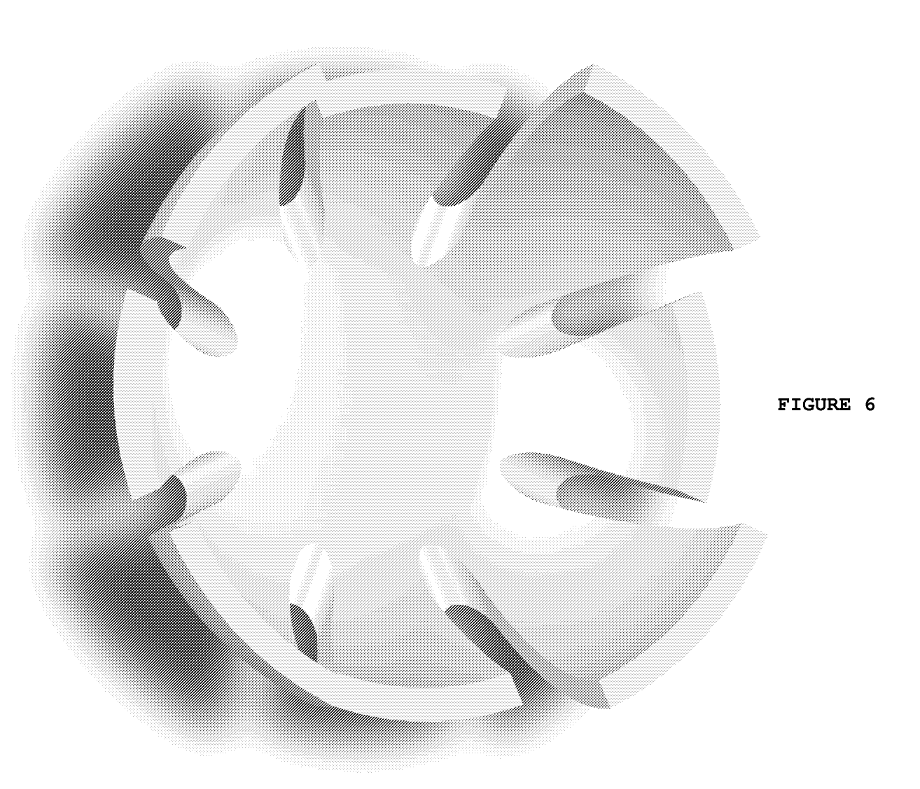

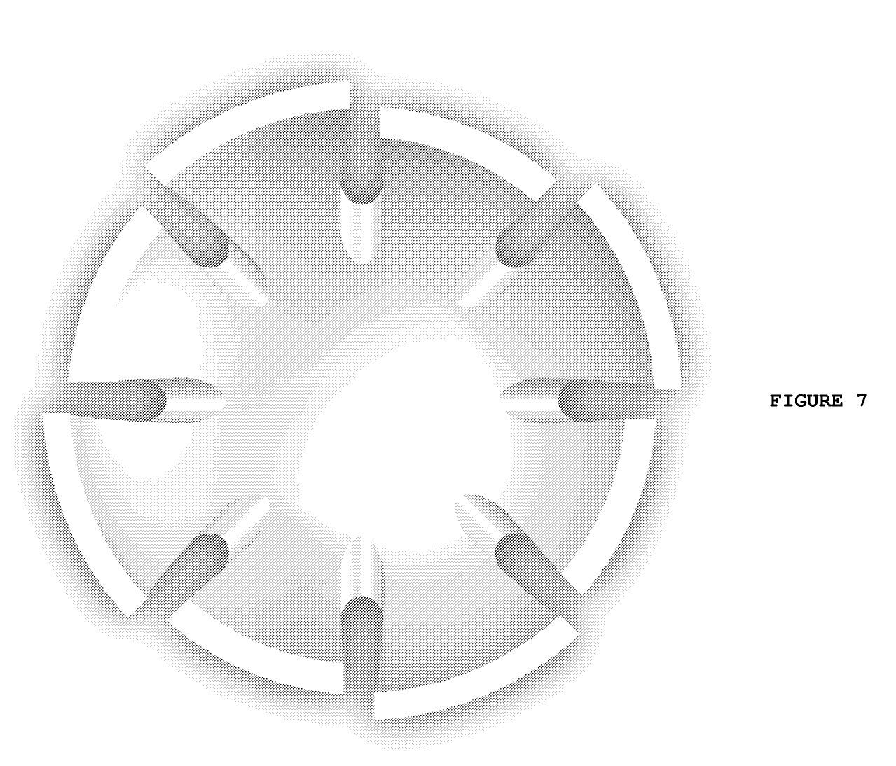

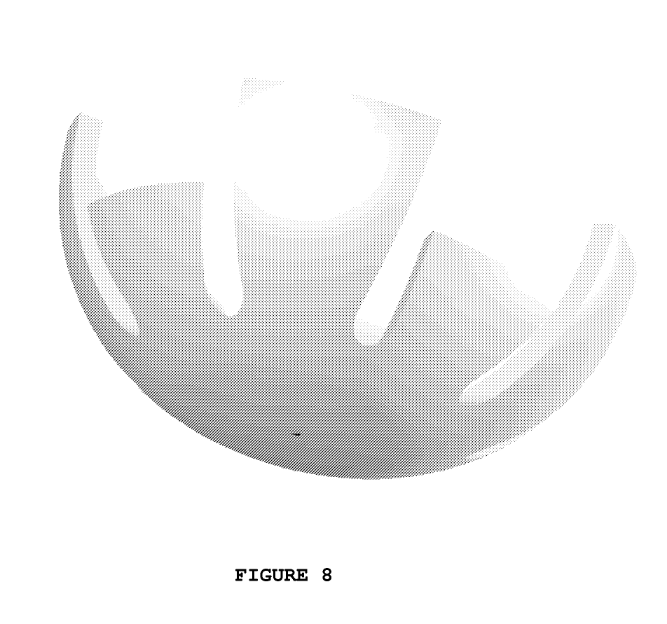

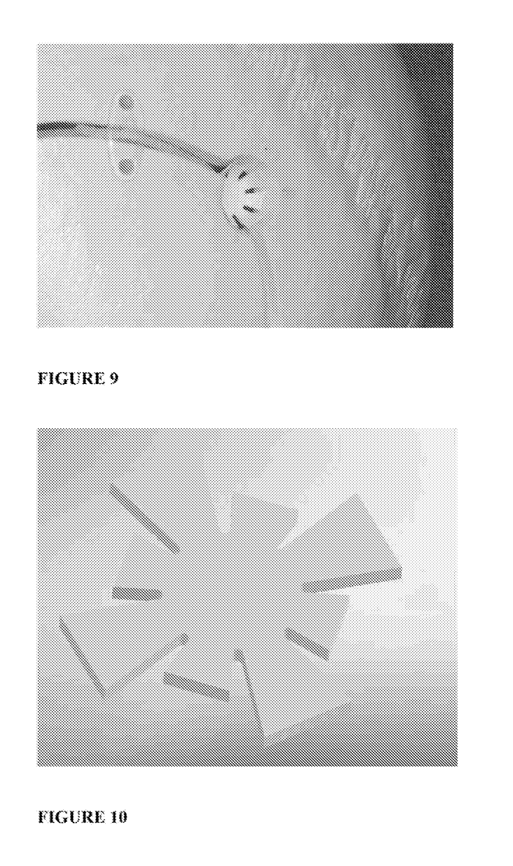

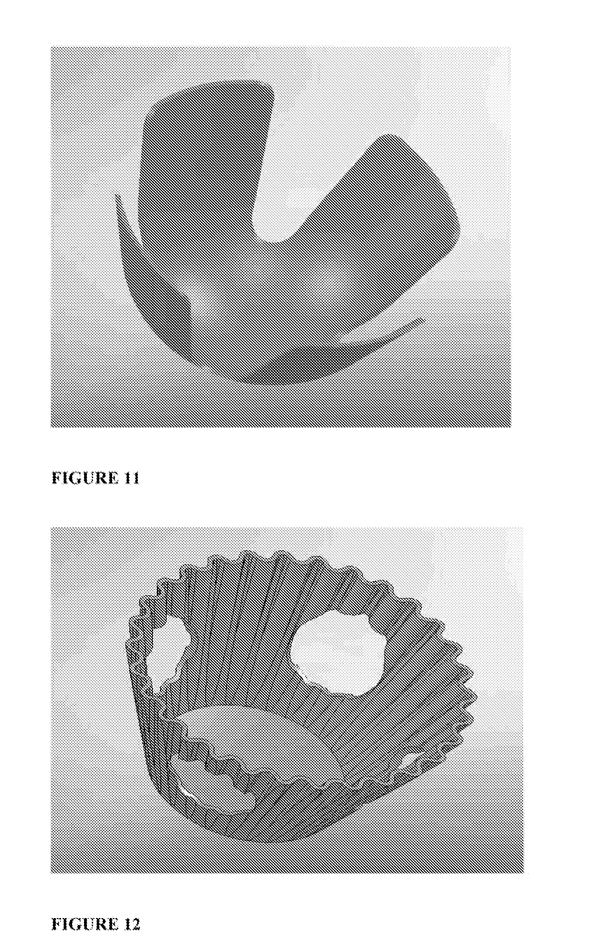

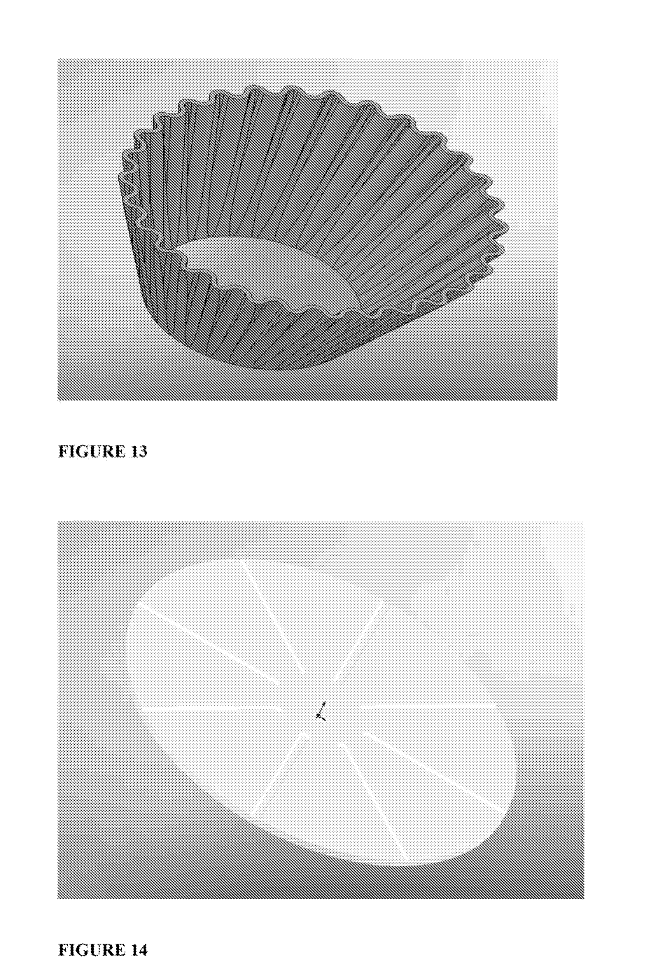

Any of the plugs disclosed in this application could be produced in either a preformed shape (e.g., FIGS. 1-8) or a flat condition (e.g., FIG. 10). Either style would produce the desired result provided the material was sufficiently flexible. In a preferred embodiment, the installed plug provides gaps, holes or slots in the sidewall to allow bone graft or bioactive materials to directly contact the sidewalls of the burr hole thereby improving fusion. Alternately the sidewalls can be solid and the plug material could be a rapidly dissolving material or a bioabsorbable material impregnated with a bioactive substance. FIGS. 12 and 13 show an embodiment with and without holes in a side wall.

In the flat condition, the plugs may be pre-scored or embossed to cause them to fold/collapse in a predetermined manner. Such an embodiment is shown in FIG. 14.

In an alternate embodiment (not shown), the cranial plug may be in essentially flat condition and tucked under the skull to cover the dura in the area(s) directly below the burr hole(s) before the skull flap is reattached. The plug/dura cover is made from a sheet of generally even thickness and can be in one piece or more than one piece. The cranial plug of this design would not be inserted into the burr hole and would not have any significant contact with the sidewalls of the burr hole.

What is claimed:

1. A cranial plug for a burr hole or a cranial perforation made in a skull during brain surgery, the cranial plug having a bottom portion and side walls extending upwards from the bottom portion, the bottom portion and the side walls forming a substantially concave shape.

2. The cranial plug of claim 1, wherein the side walls comprise at least two tabs.

3. The cranial plug of claim 2, wherein the tabs are not in contact with each other.

4. The cranial plug of claim 2, wherein the tabs have different heights.

5. The cranial plug of claim 2, wherein the tabs are the same height.

6. The cranial plug of claim 1, wherein the side walls have a top surface and wherein the top surface is not in contact with any portion of the cranial plug.

7. The cranial plug of claim 1, wherein the side walls are continuous.

8. The cranial plug of claim 7, wherein the side walls comprise at least one perforation.

9. The cranial plug of claim 1, wherein the cranial plug comprises a bioabsorbable material.

10. The cranial plug of claim 9, wherein the bioabsorbable material is selected from the group consisting of collagen, polyglycolide, poly(lactic acid), copolymers of lactic acid, glycolic acid, poly-L-lactide, poly-L-lactate, copolymers of polyethylene glycol, polybutylene terephthalate and combinations thereof.

11. The cranial plug of claim 9, wherein the bioabsorbable material is flexible.

12. The cranial plug of claim 10, wherein the bioabsorbable material is collagen.

13. A cranial plug for a burr hole or a cranial perforation made in a skull during brain surgery, the cranial plug having a bottom portion and side walls, wherein the cranial plug is substantially flat prior to insertion into a burr hole or a cranial perforation and wherein the cranial plug is substantially concave, with the side walls extending upwards from the bottom portion when the cranial plug is inserted into a burr hole or a cranial perforation.

14. The cranial plug of claim 13, wherein the side walls comprise at least two tabs.

15. The cranial plug of claim 14, wherein the tabs are not in contact with each other.

16. The cranial plug of claim 14, wherein the tabs have different heights.

17. The cranial plug of claim 14, wherein the tabs are the same height.

18. The cranial plug of claim 13, wherein the side walls have a top surface and wherein the top surface is not in contact with any portion of the cranial plug.

19. The cranial plug of claim 13, wherein the side walls are continuous.

20. The cranial plug of claim 19, wherein the side walls comprise at least one perforation.

21. The cranial plug of claim 13, wherein the cranial plug comprises a bioabsorbable material.

22. The cranial plug of claim 21, wherein the bioabsorbable material is selected from the group consisting of collagen, polyglycolide, poly(lactic acid), copolymers of lactic acid, glycolic acid, poly-L-lactide, poly-L-lactate, copolymers of polyethylene glycol, polybutylene terephthalate and combinations thereof.

23. The cranial plug of claim 21, wherein the bioabsorbable material is flexible.

24. The cranial plug of claim 22, wherein the bioabsorbable material is collagen.
